(19) United States
(12) Patent Application Publication
(10) Pub. No.: US 2026/0182845 A1
(43) Pub. Date: Jul. 2, 2026

(54) BLOOD GLUCOSE AND BLOOD PRESSURE MEASUREMENT

(71) Applicant: Accurate Meditech Inc., New Taipei City (TW)

(72) Inventor: Kuan Jen Wang, New Taipei City (TW)

(21) Appl. No.: 19/434,773

(22) Filed: Dec. 29, 2025

Related U.S. Application Data
(60) Provisional application No. 63/741,384, filed on Jan. 2, 2025.

(51) Int. Cl. A61B 5/0205; A61B 5/00; A61B 5/021; A61B 5/145

(52) U.S. Cl. CPC A61B 5/0205; A61B 5/14532; A61B 5/681; A61B 5/021; A61B 2562/0247; A61B 2562/043; A61B 2562/06

(57) ABSTRACT

A wearable device for measuring blood glucose and blood pressure for a user includes a main body including a first side and a second side formed on the opposite side of the first side; a first supporting frame protruding from the first side and a second supporting frame protruding from the second side; a belt portion having a first belt end detachably coupled to the first supporting frame, a second belt end detachably coupled to the second supporting frame, and at least one aligning mark, wherein the belt portion is positioned around a wrist of the user; wherein the main body includes at least one pressure sensor array assembly overlaid on a radial artery of the user; wherein the pressure sensor array assembly includes a plurality of apertures arranged one after another to form a cross-shaped pattern and at least four sensor elements arranged in a symmetrical configuration.

### FIELD OF THE DISCLOSURE

The present disclosure relates to a blood glucose and blood pressure measurement, and more particularly, a wearable device for measuring blood glucose and blood pressure, which has accurate results and is user-friendly.

### BACKGROUND OF THE DISCLOSURE

Generally, the rising awareness of health, monitoring blood glucose and blood pressure has become an essential part of daily health management. A blood glucose and blood pressure monitor is a multifunctional health device that helps users quickly and accurately measure their blood glucose and blood pressure levels, enabling effective health tracking. The current blood glucose and blood pressure monitor has a dual function integration: blood glucose and blood pressure monitors combine both functionalities and saving the cost of purchasing multiple devices. They are suitable for home use or portability.

However, monitoring the current blood glucose and blood pressure has disadvantages. For example, the measurements may not always be as accurate as professional medical-grade equipment, and factors like user technique, body movement, or device calibration can introduce errors. In addition, the current blood glucose and blood pressure monitor requires regular calibration and proper care to maintain accuracy.

Blood glucose and blood pressure monitors have significantly improved health management, but there is still room for advancement. Enhancing accuracy, usability, affordability, and accessibility will make these devices more effective and user-friendly.

### BRIEF SUMMARY OF THE DISCLOSURE

In a general implementation, the wearable device for measuring blood glucose and blood pressure for a user may comprise a main body comprising a first side and a second side formed on the opposite side of the first side; a first supporting frame protruding from the first side and a second supporting frame protruding from the second side formed on the opposite side of the first side; a belt portion having a first belt end detachably coupled to the first supporting frame, a second belt end detachably coupled to the second supporting frame, and at least one aligning mark, wherein the belt portion is positioned around a wrist of the user; wherein the main body comprises at least one pressure sensor array assembly overlaid on a radial artery of the user; wherein the pressure sensor array assembly comprises a plurality of apertures arranged one after another and in elongated linear arrays extending along at least a first direction and a second direction intersecting the first direction to form a cross-shaped pattern and at least four sensor elements arranged in a symmetrical configuration and spatially separated by the cross-shaped apertures; wherein each one of at least four sensor elements doesn't contact each other.

In another aspect combinable with the general implementation, the pressure sensor array assembly may comprise a substrate having a first portion, a second portion formed on the opposite side of the first portion, and an attaching cavity formed between the first portion and the second portion.

Further, it is contemplated that the belt portion is inserted inside the attaching cavity to secure the pressure sensor array assembly on the belt portion.

In the alternative, the plurality of apertures and the at least four sensor elements are arranged on the second portion of the substrate without being arranged on the first portion of the substrate.

It is still further contemplated that the second portion of the substrate is divided into four sensor areas by the cross-shaped apertures, wherein each of the four sensor areas consists of only one of the at least four sensor elements.

In another aspect combinable with the general implementation, the aligning mark is an aligning line configured to align with an interspace between a ring finger of the user and a pinky finger of the user.

In another aspect combinable with the general implementation, the aligning mark is an arrow mark configured to align with a middle portion of a wrist of the user.

In another aspect combinable with the general implementation, the wearable device further may comprise an attaching assembly which is secured on the belt portion and comprises a male magnet, wherein the pressure sensor array assembly comprises a substrate having a female magnet, wherein the female magnet is attracted by the male magnet of the attaching assembly to magnetically secure the substrate with the belt portion.

In another aspect combinable with the general implementation, the wearable device may further comprise an attaching assembly coupled with the first supporting frame, wherein the attaching assembly is an elongated strap having a first attaching unit end securely coupled to the first supporting frame and a second attaching unit end securely coupled with the pressure sensor array assembly, wherein the first attaching unit end is formed on an opposite end of the second attaching unit end.

In another aspect combinable with the general implementation, the first attaching unit end is securely sandwiched between the first supporting frame and the belt portion, wherein the first supporting frame comprises a first locking cavity where the first attaching unit end is received therein.

In another aspect combinable with the general implementation, the first attaching unit end is a curved shape to form a second locking cavity, wherein the belt portion is received inside the second locking cavity and is coupled to the first supporting frame to secure the first attaching unit end with the first supporting frame.

Another aspect of the embodiment is directed to methods of measuring blood glucose and blood pressure of a user by using a wearable device, comprising the steps of:
providing a wearable device comprising:
a main body having a first side, a second side formed on an opposite side of the first side, and at least one pressure sensor array assembly;
a first supporting frame protruding from the first side and a second supporting frame protruding from the second side;
a belt portion having a first belt end detachably coupled to the first supporting frame, a second belt end detachably coupled to the second supporting frame, and at least one aligning mark;
positioning the belt portion around a wrist of the user;
overlaying the at least one pressure sensor array assembly on a radial artery of the user; wherein
the pressure sensor array assembly comprises a substrate having a plurality of apertures arranged one after another and in elongated linear arrays extending along at least a first direction and a second direction intersecting the first direction to form cross-shaped pattern and at least four sensor elements arranged in a symmetrical configuration and spatially separated by the cross-shaped apertures; wherein each one of at least four sensor elements doesn't contact each other.

In another aspect combinable with the general implementation, the method may further comprise steps of:
inserting the belt portion inside an attaching cavity of a substrate of the pressure sensor array assembly, wherein the substrate comprises a first portion, a second portion formed on an opposite side of the first portion, and an attaching cavity formed between the first portion and the second portion, wherein the plurality of apertures and the at least four sensor elements are arranged on the second portion of the substrate.

In another aspect combinable with the general implementation, the method may further comprise steps of:
aligning an aligning mark comprising an aligning line with an interspace between a ring finger and a pinky finger of the user.

In another aspect combinable with the general implementation, the method may further comprise steps of:
aligning an arrow mark formed on the belt portion with a middle portion of the wrist of the user to ensure proper positioning of the pressure sensor array assembly on a radial artery of the user.

In another aspect combinable with the general implementation, the method may further comprise steps of:
magnetically securing the pressure sensor array assembly to the belt portion by attracting a female magnet on the substrate toward a male magnet disposed on an attaching assembly secured on the belt portion.

In another aspect combinable with the general implementation, the method may further comprise steps of:
coupling a first attaching unit end of an attaching assembly to the first supporting frame, wherein the attaching assembly is an elongated strap having the first attaching unit end and a second attaching unit end; and
coupling a second attaching unit of the attaching assembly with the pressure sensor array assembly.

In another aspect combinable with the general implementation, the method may further comprise steps of:
receiving the first attaching unit end inside a first locking cavity of the first supporting frame; and
sandwiching the first attaching unit end between the first supporting frame and the belt portion to secure the first attaching unit end of attaching assembly with the first supporting frame.

In another aspect combinable with the general implementation, the method may further comprise steps of:
receiving a first belt end of the belt portion inside a second locking cavity formed by the first attaching unit end of the attaching assembly; and
sandwiching the first attaching unit end between the first supporting frame and the belt portion to secure the first attaching unit end of the attaching assembly with the first supporting frame.

Among the many possible implementations of the pressure sensor array assembly for detachably coupled on a belt portion of a wearable device, the pressure sensor array assembly may comprise an attaching unit comprising a male magnet and detachably coupled with the belt portion and a female magnet matching with the male magnet; a substrate having a first portion and a second portion formed on an opposite side of the first portion; a plurality of apertures arranged one after another to form cross-shaped apertures and arranged in the first portion; and a plurality of sensor elements separated by the cross-shaped apertures and arranged in the first portion; wherein the substrate is made of magnetic material and configured to attract the female magnet fixedly.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above and below as acting in certain combinations and even initially claimed as such, one or more features from a claimed combination can in some cases be excised from the combination, and the claimed combination may be directed to a subcombination or variation of a subcombination. A number of implementations have been described. Nevertheless, it will be understood that various modifications may be made without departing from the spirit and scope of the disclosure. For example, the operations, methods, or processes described herein may include more or fewer steps than those described. Further, the steps in such example operations, methods, or processes may be performed in different successions than that described or illustrated in the figures. Accordingly, other implementations are within the scope of the following claims.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

It should be noted that the drawing figures may be in simplified form and might not be of a precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1A is a top view of the main body of the wearable device according to an aspect of the embodiment.

FIG. 1B is a bottom view of the main body of the wearable device according to an aspect of the embodiment.

FIG. 2 is a perspective view of the wearable device according to an aspect of the embodiment.

FIG. 3 shows a belt portion of the wearable device according to an aspect of the embodiment.

FIG. 4A is a top view of the belt portion of the wearable device according to an aspect of the embodiment.

FIG. 4B is a front view of the belt portion of the wearable device according to an aspect of the embodiment.

FIG. 5 shows the wearable device worn on the wrist, illustrating the blood pressure measurement according to an aspect of the embodiment.

FIG. 6 is a perspective view showing the wearable device worn on the wrist, illustrating an aligning mark aligning with a middle of the wrist of the user, according to an aspect of the embodiment.

FIG. 7A is a front view of a pressure sensor array assembly according to an aspect of the embodiment.

FIG. 7B is a top view of the pressure sensor array assembly according to an aspect of the embodiment.

FIG. 8A shows the pressure sensor array assembly attached to a belt portion of the wearable device according to an aspect of the embodiment.

FIG. 8B is a side view of the pressure sensor array assembly attached to a belt portion of the wearable device according to an aspect of the embodiment.

FIG. 9 is a perspective view showing the wearable device worn on the wrist, illustrating an aligning mark aligning with a middle of the wrist of the user, according to another aspect of the embodiment.

FIG. 10 is a perspective view showing the pressure sensor array assembly being coupled with the belt portion according to still another aspect of the embodiment.

FIG. 11A is a front view of a pressure sensor array assembly according to still another aspect of the embodiment.

FIG. 11B is a top view of the pressure sensor array assembly according to still another aspect of the embodiment.

FIG. 12 is a perspective view of the pressure sensor array assembly being charged in the charging box according to still another aspect of the embodiment.

FIG. 13 is a perspective view showing the wearable device worn on the wrist, illustrating an aligning mark aligning with the middle of the wrist of the user, according to still another aspect of the embodiment.

FIG. 14 is a perspective view of the pressure sensor array assembly being attached to a first supporting frame of the wearable device according to still another aspect of the embodiment.

FIG. 15 is a sectional view showing an attaching unit of the pressure sensor array assembly being attached to the first supporting frame of the wearable device according to still another aspect of the embodiment.

FIG. 16 is a bottom view of the pressure sensor array assembly being attached to the first supporting frame of the wearable device according to still another aspect of the embodiment.

FIG. 17 shows a side sectional view showing the belt portion and the attaching assembly attached to the first supporting frame according to still another aspect of the embodiment.

FIG. 18 is a sectional view showing a belt portion being attached to the first supporting frame of the wearable device according to still another aspect of the embodiment.

FIG. 19 is another perspective view showing the wearable device worn on the wrist, illustrating an aligning mark aligning with the middle of the wrist of the user, according to still another aspect of the embodiment.

### DETAILED DESCRIPTION OF THE EMBODIMENTS

The different aspects of the various embodiments can now be better understood by turning to the following detailed description of the embodiments, which are presented as illustrated examples of the embodiments defined in the claims. It is expressly understood that the embodiments as defined by the claims may be broader than the illustrated embodiments described below.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

Unless defined otherwise, all technical and position terms used herein have the same meaning as commonly understood by one of ordinary skill in the art to which the invention pertains. Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

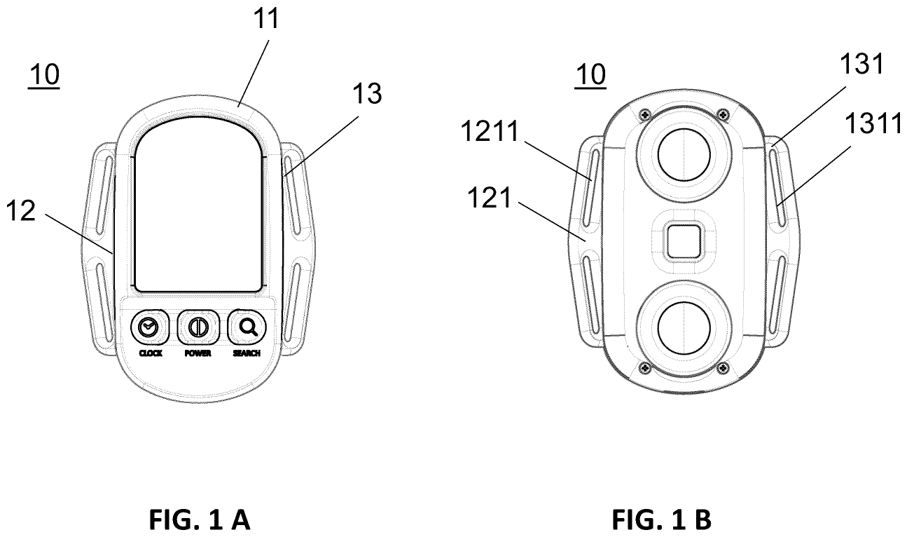

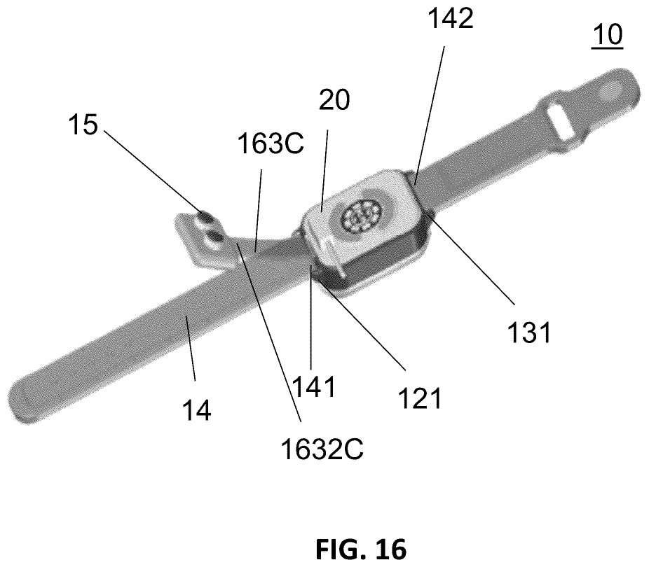

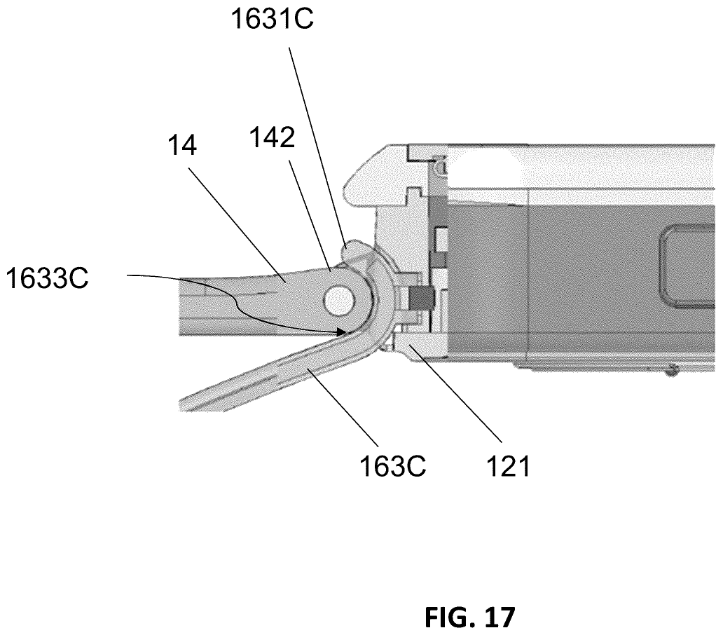

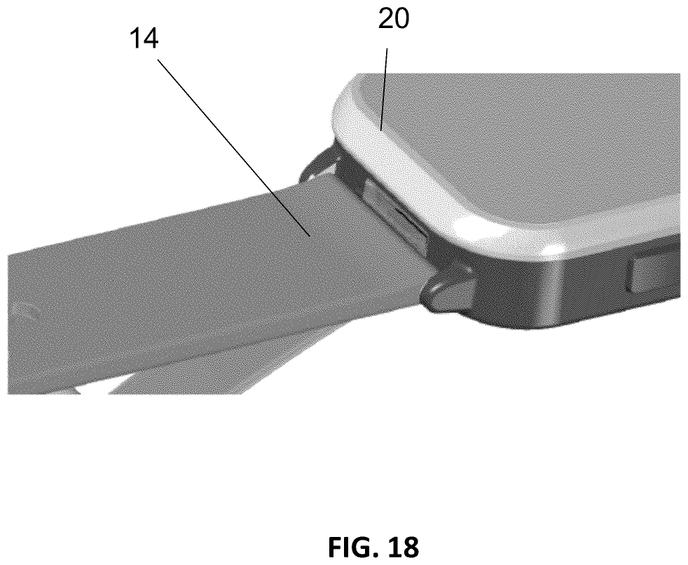

FIG. 1A to FIG. 19 generally depict a wearable device 10 for measuring blood glucose and blood pressure according to an aspect of the embodiment.

Referring to FIG. 1A and FIG. 1B, the wearable device 10 may comprise a main body 11 comprising a first side 12 and a second side 13 formed on the opposite side of the first side 12, a first supporting frame 121 protruding from the first side 12 of the main body 11, and a second supporting frame 131 protruding from the second side 13 of the main body 11.

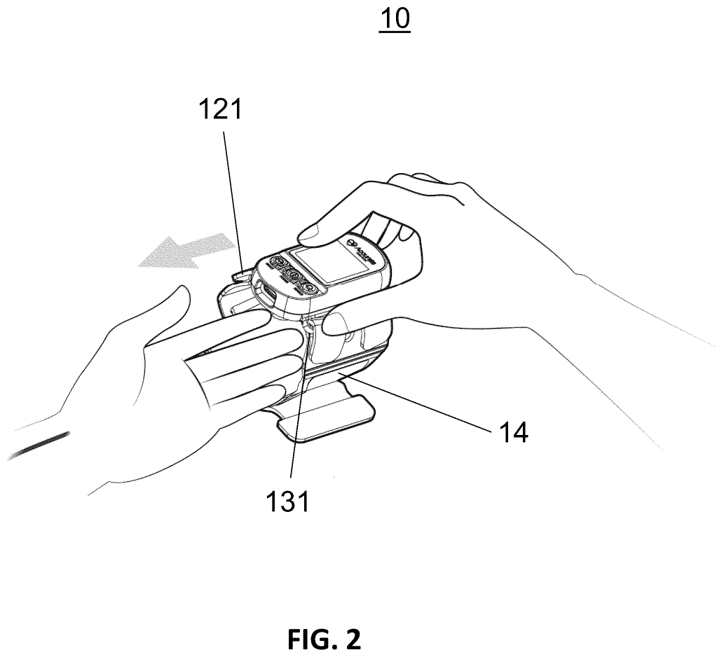

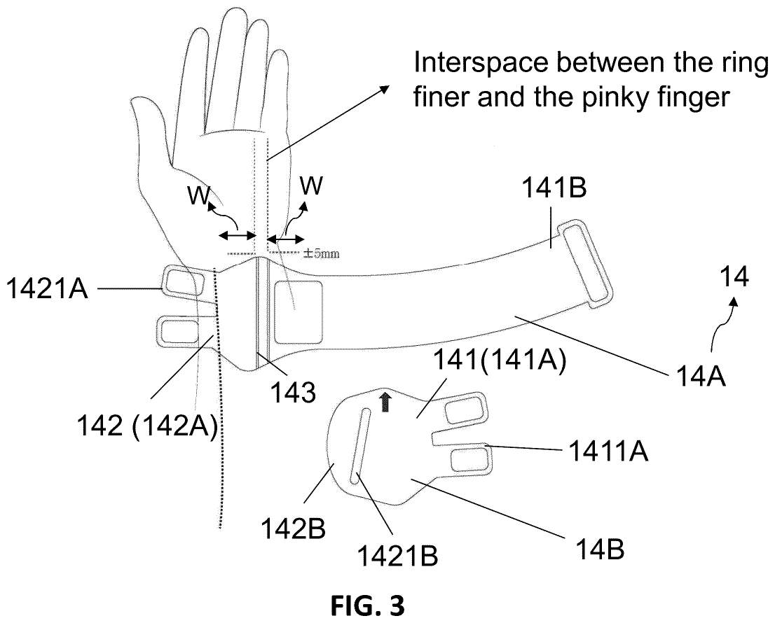

Referring to FIG. 2 and FIG. 3, in some embodiments, the wearable device 10 may comprise a belt portion 14 having a first belt end 142 detachably coupled to the first supporting frame 121 and a second belt end 141 detachably coupled to the second supporting frame 131, and at least one aligning mark 143 configured to align with an interspace between a ring finger of the user and a pinky finger of the user. In other words, the belt portion 14 may be positioned around the user's wrist.

Referring to FIGS. 2, 3, FIG. 4A, and FIG. 4B, the belt portion 14 may comprise an elongated primary main belt portion 14A having a first primary belt end 142A detachably coupled to the first supporting frame 121 and a secondary primary belt end 141B formed on the opposite side of the first primary belt end 142A. The wearable device 10 may further comprise a secondary main belt portion 14B having a first-secondary main belt portion end 141A detachably coupled to the second supporting frame 131 and a second-secondary main belt portion end 142B formed on the opposite side of the first secondary main belt portion 141A.

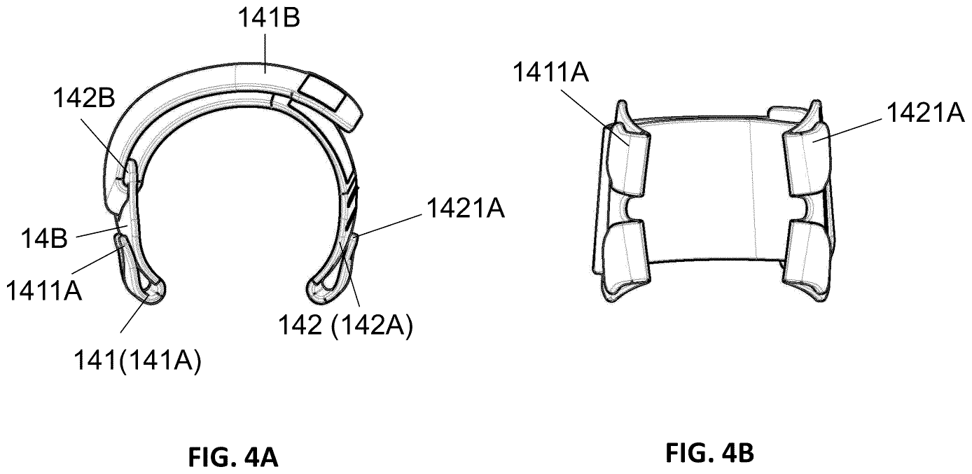

In some embodiments, referring to FIG. 3, FIG. 4A, and FIG. 4B, the first primary belt end 142A may be integrally protruded from the elongated main belt portion 14A to form a first locking member 1421A. Preferably, the first primary belt end 142A may comprise a pair of first locking members 1421A integrally protruding from the elongated primary main belt portion 14A. In some embodiments, the first secondary main belt end 141A may be integrally protruding from the secondary main belt portion 14B to form a second locking member 1411A. Preferably, the first secondary belt end 141A may comprise a pair of second locking members 1411A integrally protruding from the secondary main belt portion 14B.

It should be noted that, in some embodiments, the second secondary main belt portion end 142B may comprise a secondary main belt hole 1421B, wherein secondary primary belt end 141B may pass through the secondary main belt hole 1421B to couple with the elongated primary main belt portion 14A, and in such a way, the elongated primary main belt portion 14A may be coupled with the secondary main belt portion 14B. Referring back to FIG. 1A, FIG. 1B, and FIG. 3, in some embodiments, the first supporting frame 121 may comprise at least one first securing hole 1211 configured to receive the belt portion 14, and the second supporting frame 131 may comprise at least one second securing hole 1311 configured to receive the belt portion 14. Preferably, the first supporting frame 121 may comprise at least two first securing holes 1211 configured to receive the belt portion 14. Preferably, the second supporting frame 131 may comprise at least two second securing holes 1311 configured to receive the belt portion 14.

In some embodiments, each of the first locking members 1421A may pass through each of the first securing holes 1211 and to attach on the elongated primary main belt portion 14A, and each of the second locking members 1411A may pass through each of the second securing holes 1311 and to attach on the secondary main belt portion 14B, and in such a way, the main body 11 of the wearable device 10 may be fixedly attached with the belt portion 14.

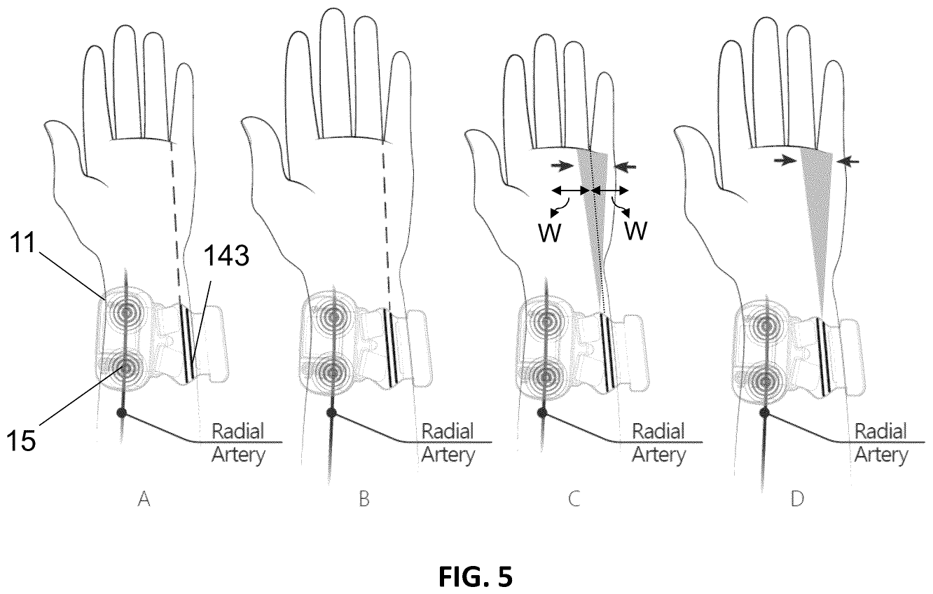

Referring to FIG. 5, it should be noted that, in some embodiments, the main body 11 may further comprise at least one pressure sensor array assembly 15 overlaying a radial artery of the user, wherein while the aligning mark 143 is aligned within the interspace defined by extending a distance “W” from the right side and the left side of the webbing between the ring finger of the user and the pinky finger of the user respectively, the pressure sensor array assembly 15 may be configured to cover the radial artery of the user, and at such a situation, the pressure sensor array assembly 15 may function efficiently in order to obtain the best results.

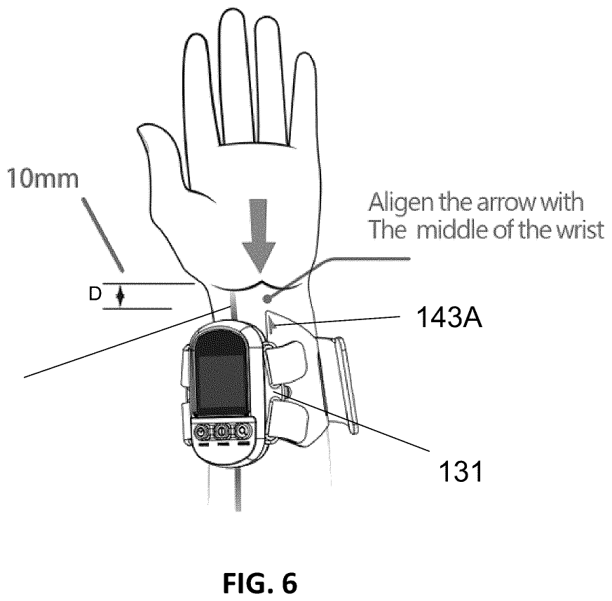

Referring to FIG. 6, in some embodiments, the aligning mark 143 may be an arrow mark 143A configured to align with a middle portion of the wrist of the user. It should be noted that, in some embodiments, the arrow mark 143A may be disposed at a predetermined distance of about 10 mm from the middle of the wrist of the user in order to obtain the best results measuring by the pressure sensor array assembly 15.

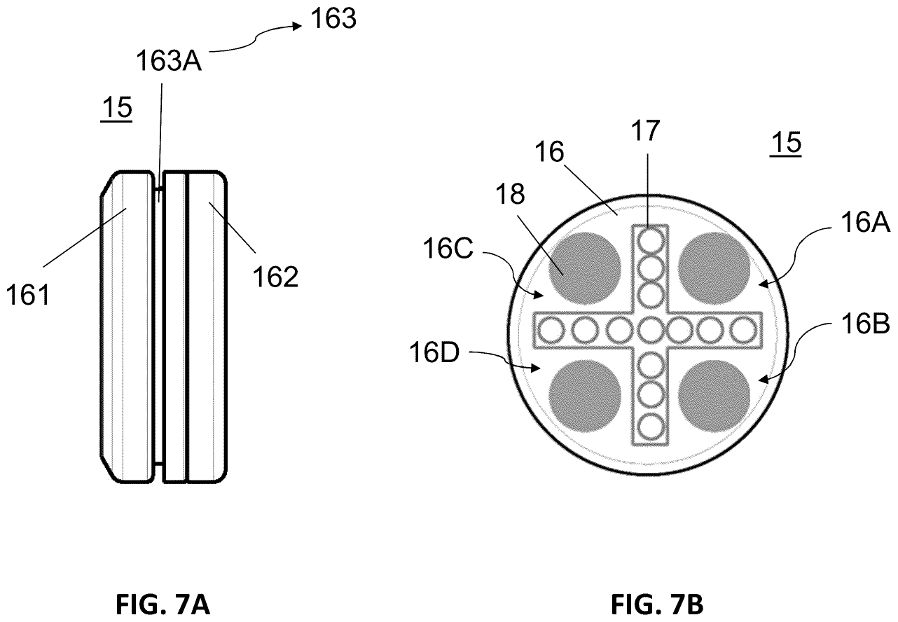

Referring to FIG. 7A and FIG. 7B, in some embodiments, the pressure sensor array assembly 15 may comprise a substrate 16 having a plurality of apertures 17 embedded thereon and arranged one after another to form a cross-shape, wherein the substrate 16 may further comprise at least four sensor elements 18 embedded thereon and separated by the cross-shaped apertures 17, wherein the substrate 16 may be divided into four sensor areas, 16A, 16B, 16C, and 16D by the cross-shaped apertures 17, and each of the sensor areas 16A, 16B, 16C, and 16D comprises only one sensor elements 18 to be separated inside each of the four sensor areas 16A, 16B, 16C, and 16D.

It should be noted that, in some embodiments, referring to FIG. 7A and FIG. 7B, the pressure sensor array assembly 15 may comprise the substrate 16 having a first portion 161 and a second portion 162 formed on an opposite side of the first portion 161, a plurality of apertures 17 arranged one after another to form a cross-shape and arranged in the second portion 162, and the at least four sensor elements 18 separated by the cross-shaped apertures 17 and arranged in the second portion 162.

It should be noted that, in some embodiments, the plurality of apertures 18 may be arranged one after another and in elongated linear arrays extending along at least a first direction and a second direction intersecting the first direction to form a cross-shaped pattern and at least four sensor elements 18 arranged in a symmetrical configuration and spatially separated by the cross-shaped apertures 17.

As shown in further details of FIG. 7A and FIG. 7B, in some embodiments, the second portion 162 of the substrate 16 may be divided into four sensor areas 16A, 16B, 16C, and 16D by the cross-shaped apertures 17, and each of the four sensor areas 16A, 16B, 16C, 16D comprises only one sensor element 18 to be separated inside each of the four sensor areas 16A, 16B, 16C, 16D. In other words, each one of the plurality of sensor elements 18 does not contact each other.

It should be noted that, in some embodiments, the plurality of apertures 17 and the at least four sensor elements 18 may be only arranged on the second portion 162 of the substrate 16 without being arranged on the first portion 161 of the substrate 60.

In some embodiments, the wearable device 10 may further comprise an attaching assembly 163, wherein in some embodiments, the attaching assembly 163 may be an attaching cavity 163A formed between the first portion 161 of the substrate 16 and the second portion 162 of the substrate 16 and configured to be detachably affixed on the belt portion 14 of the wearable device, wherein the attaching assembly 163 may be inwardly extended from an outer surface of the substrate 16 to form the attaching cavity 163A where the belt portion 14 of the wearable device is received therein, and in such a situation, the pressure sensor array assembly 15 may be securely attached on the belt portion 14.

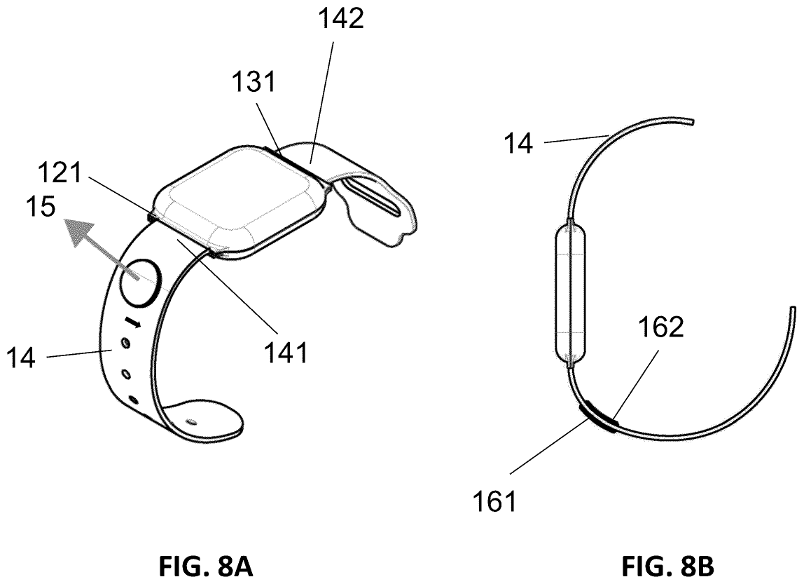

As shown in FIG. 8A and FIG. 8B, the belt portion 14 may be inserted inside the attaching cavity 163A to secure the pressure sensor array assembly 15 on the belt portion 14.

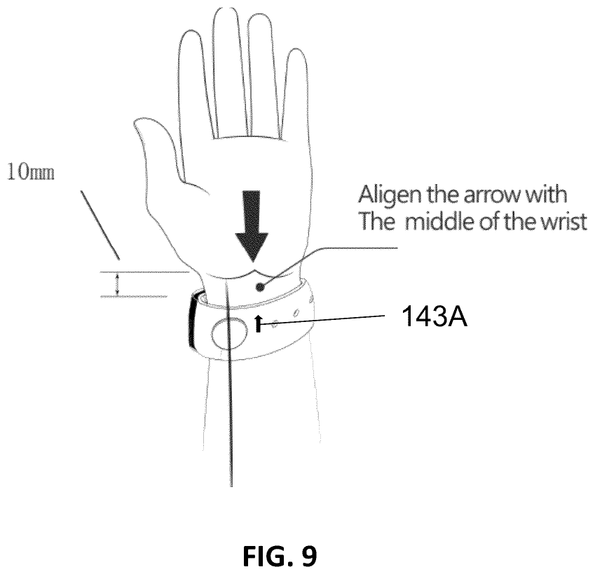

As shown in FIG. 9, in some embodiments, the aligning mark 143 may be the arrow mark 143A, configured to align with the middle portion of the user's wrist. It should be noted that, in some embodiments, the arrow mark 143A may be disposed at a predetermined distance of about 10 mm from the middle of the wrist in order to obtain the best results measuring by the pressure sensor array assembly 15.

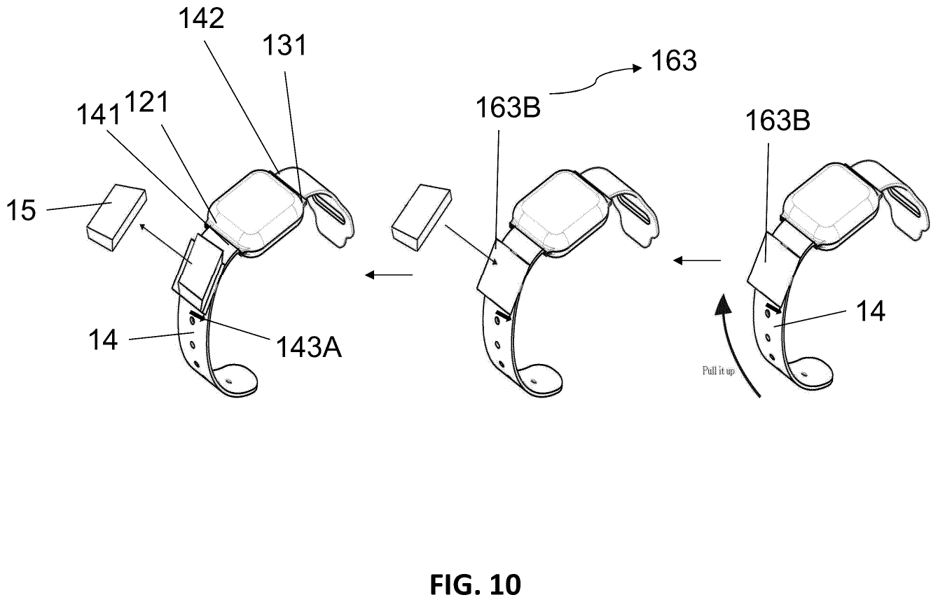

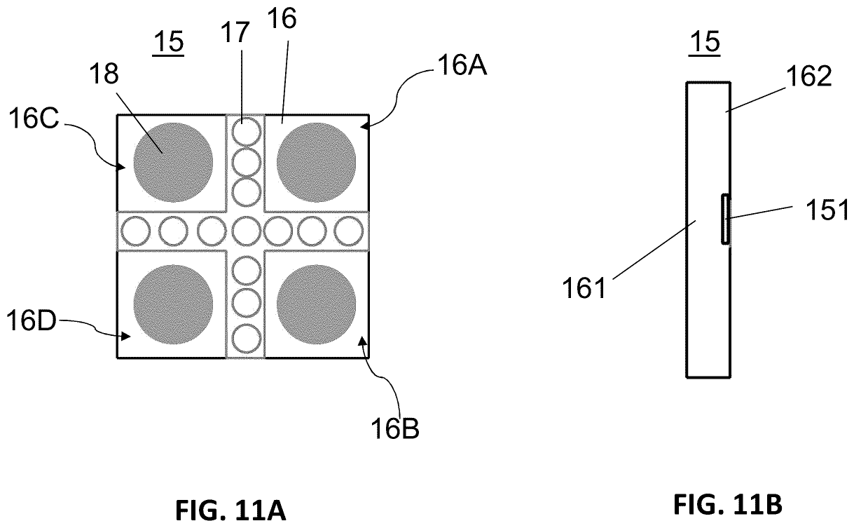

Referring to FIGS. 10-11B, in some embodiments, the wearable device 10 may further comprise the attaching assembly 163 detachably coupled with the belt portion 14, wherein the attaching assembly 163 may be a male magnet 163B configured to be attracted with a female magnet 151 formed on the substrate 16, wherein the female magnet 151 may be securely attached to the male magnet 163B. In other words, the substrate 16 having the female magnet 151 may be attracted by the male magnet 163B to magnetically secure the substrate 16 with the belt portion 14.

It should be noted that, in some embodiments, the substrate 16 may be made of magnetic materials, such as iron, nickel, cobalt, steel (which contains iron), and certain alloys like alnico and rare-earth magnets (such as neodymium-iron-boron) and configured to be attracted by the male magnet 163B fixedly.

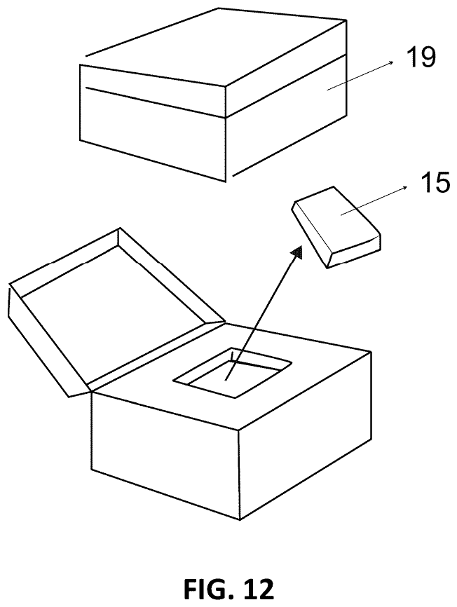

Continuing to FIG. 12, the pressure sensor array assembly 15 may be charged by a charging box 19, and in such a way, the pressure sensor array assembly 15 may be arranged inside the charging box 19 to recharge the pressure sensor array assembly 15.

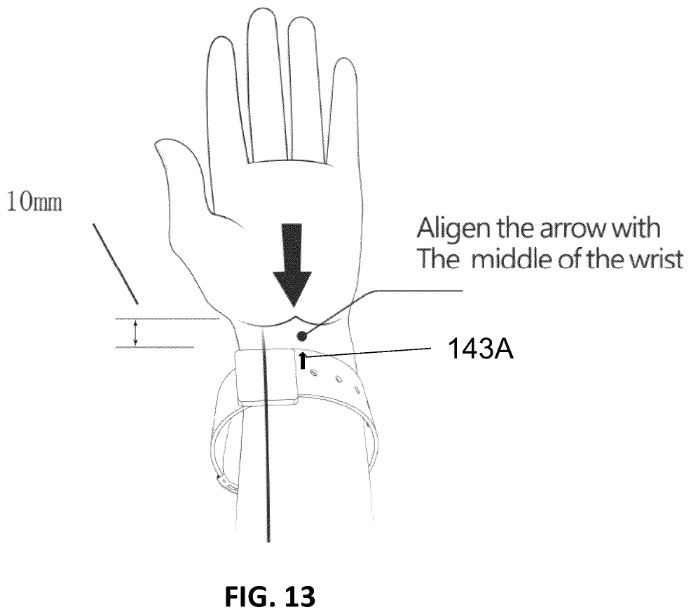

As shown in FIG. 13, in still some embodiments, the aligning mark 143 may be the arrow mark 143A configured to align with the middle portion of the wrist of the user.

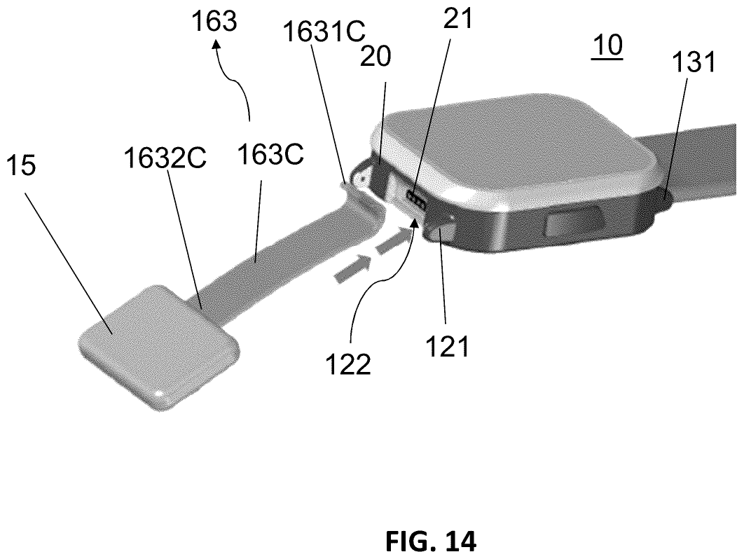

Referring to FIGS. 14-16, the wearable device may further comprise the attaching assembly 163 coupled with the first supporting frame 121, wherein the attaching assembly 163 may be an elongated strap having a first attaching unit end 1631C securely coupled to the first supporting frame 121 and a second attaching unit end 1632C securely coupled with the pressure sensor array assembly 15, and in such a way, the first attaching unit end 1631C may be formed on an opposite side of the second attaching unit end 1632C.

As shown in further details in FIGS. 14-16, the first attaching unit end 1631C may be formed as a curved shape to form a second locking cavity 1633C, wherein the first attaching unit end 1631C may be securely sandwiched between the first supporting frame 121 and the belt portion 14, and in such a way, the first attaching unit end 1631C may be securely coupled with the first supporting frame 121. In other words, the belt portion 14 may be received inside the second locking cavity 1633C and configured to bias/press against the first attaching unit 1631C with the first supporting frame 121.

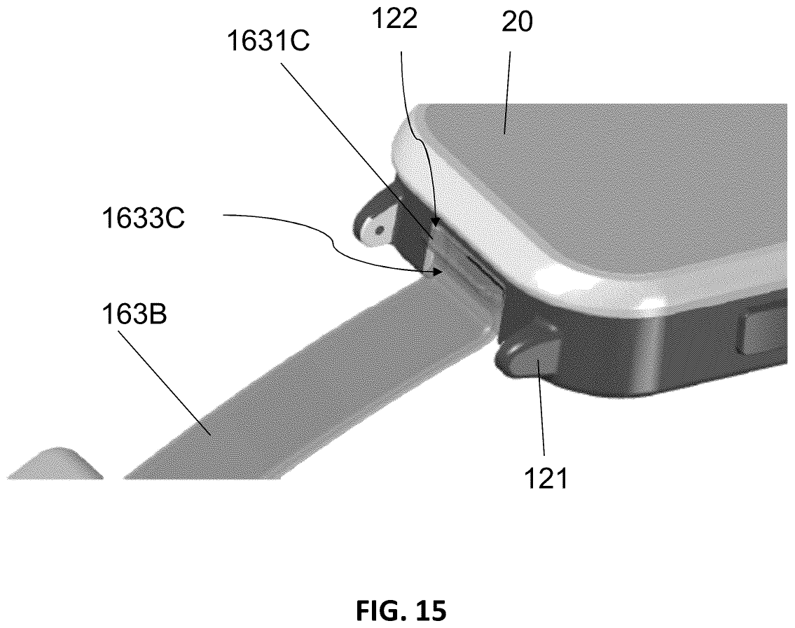

Continuing to FIGS. 14 and 15, the first supporting frame 121 may further comprise a first locking cavity 122, and the first attaching unit end 1631C may be received inside the first locking cavity 122, and at the same time, the belt portion 14 may be coupled with the first supporting frame 121, and in other words, the first attaching unit end 1631C may be securely sandwiched between the first supporting frame 121 and the belt portion 14.

Referring to FIGS. 15-18, in some embodiments, the first attaching unit end 1631C may be a curved shape to form a second locking cavity 1633C, wherein the first belt end 142 of the belt portion 14 may be received inside the second locking cavity 1633C and configured to secure the first attaching unit end 1631C with the first supporting frame 121. In other words, the first attaching unit end 1631C may be securely sandwiched between the first supporting frame 121 and the first belt end 142 of the belt portion 14.

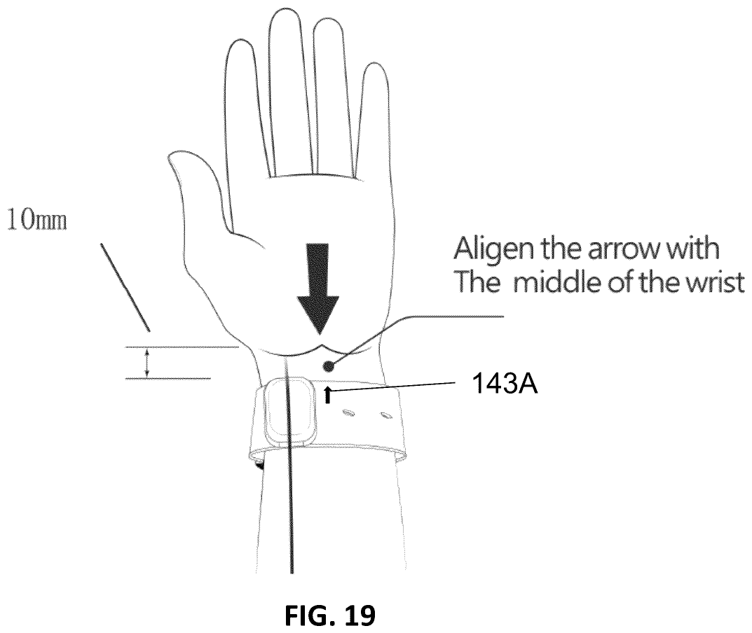

As shown in FIG. 19, in still some embodiments, the aligning mark 143 may be the arrow mark 143A configured to align with the middle portion of the wrist of the user. It should be noted that, in some embodiments, the arrow mark 143A may be disposed at a predetermined distance of about 10 mm from the middle of the wrist in order to obtain the best results measuring by the pressure sensor array assembly 15.

In still some embodiments, a method for measuring blood glucose and blood pressure of a user by using a wearable device 10, comprising the steps of:
providing a wearable device 10 comprising a main body 10 having a first side 12, a second side 13 formed on an opposite side of the first side 12, and at least one pressure sensor array assembly 15; a first supporting frame 121 protruding from the first side 12 and a second supporting frame 131 protruding from the second side 13; a belt portion 14 having a first belt end 142 detachably coupled to the first supporting frame 121, a second belt end 141 detachably coupled to the second supporting frame 131, and at least one aligning mark 143;
positioning the belt portion 14 around the wrist of the user; and
overlaying the at least one pressure sensor array assembly 15 on a radial artery of the user; wherein
the pressure sensor array assembly 15 comprises a substrate 16 having a plurality of apertures 17 arranged one after another and in elongated linear arrays extending along at least a first direction and a second direction intersecting the first direction to form a cross-shaped pattern and at least four sensor elements 18 arranged in a symmetrical configuration and spatially separated by the cross-shaped apertures.

The method for measuring blood glucose and blood pressure of the user by using the wearable device 10 may further comprise steps of:
inserting the belt portion 14 inside an attaching cavity 163A of a substrate 16 of the pressure sensor array assembly 15, wherein the substrate 16 comprises a first portion 161, a second portion 162 formed on an opposite side of the first portion 161, and the attaching cavity 163A formed between the first portion 161 and the second portion 162, wherein the plurality of apertures 17 and sensor elements 18 are arranged on the second portion 162 of the substrate 16.

In some embodiments, the method for measuring blood glucose and blood pressure of the user by using the wearable device 10 may further comprise steps of:
aligning an aligning mark 163A comprising an aligning line with an interspace between a ring finger and a pinky finger of the user.

In still some embodiments, the method for measuring blood glucose and blood pressure of the user by using the wearable device 10 may further comprise steps of:
aligning an arrow mark 163A on the belt portion 14 with a middle portion of the wrist of the user to ensure proper positioning of the pressure sensor array assembly 15.

In still some embodiments, the method for measuring blood glucose and blood pressure of the user by using the wearable device 10 may further comprise steps of:
magnetically securing the pressure sensor array assembly 15 with the belt portion 14 by attracting a female magnet 151 on the substrate 16 toward a male magnet 163B disposed on an attaching assembly 163 secured on the belt portion 14.

In still some embodiments, the method for measuring blood glucose and blood pressure of the user by using the wearable device 10 may further comprise steps of:
coupling a first attaching unit end 1631C of the attaching assembly 163 with the first supporting frame 121, wherein the attaching assembly 163 may be an elongated strap having the first attaching unit end 1631C and a second attaching unit end 1632C; and
coupling the second attaching unit end 1632C with the pressure sensor array assembly 163.

In still some embodiments, the method for measuring blood glucose and blood pressure of the user by using the wearable device 10 may further comprise steps of:
receiving the first attaching unit end 1631C inside a first locking cavity 122 of the first supporting frame 121; and
sandwiching the first attaching unit end 1631C between the first supporting frame 121 and the belt portion 14 to secure the attaching assembly 163 with the first supporting frame 121.

In still some embodiments, the method for measuring blood glucose and blood pressure of the user by using the wearable device 10 may further comprise steps of:
receiving a first belt end 142 of the belt portion 14 inside a second locking cavity 1633C formed by the first attaching unit end 1631C of the attaching assembly 163; and
securing the first belt end 142 of the belt portion 14 with the first supporting frame 121; and
sandwiching the first attaching unit end 1631C of the attaching assembly 163 between the first belt end 142 of the belt portion 14 and the first supporting frame 121.

In still some embodiments, the second portion 162 of the substrate 16 may be divided into four sensor areas 16A, 16B, 16C, and 16D by the cross-shaped apertures 17, wherein each of the four sensor areas 16A, 16B, 16C, and 16D consists of only one of the plurality of sensor elements 18, wherein each one of the at least four sensors elements 18 doesn't contact each other.

Similarly, while operations and/or methods may be depicted in a particular order, this should not be understood as requiring that such operations be performed in the particular order shown or in sequential order or that all illustrated operations and/or method steps be performed to achieve desirable results. In certain circumstances, multitasking and parallel processing may be advantageous.

### Estimate Pre-Ejection Period (PEP) by Pulse Arrival Time (PAT) and Local Pulse Transit Time (LPTT)

The pre-ejection period (PEP) is the phase of the cardiac cycle between the onset of ventricular depolarization (marked by the QRS complex on an electrocardiogram) and the opening of the aortic valve. It represents the time during which the heart's ventricles are contracting (isovolumetric contraction) but have not yet ejected blood because the pressure in the ventricles has not exceeded that in the aorta or pulmonary artery.

Pulse Arrival Time (PAT) refers to the measurement of the time interval between a pulse being generated (such as from a heartbeat) and its arrival at a particular sensing point in the body (like the wrist or ankle). It is commonly used in medical settings to assess cardiovascular health.

The formula for calculating PAT is as follows:

$$PAT = PEP + PTT$$

wherein,

$$PTT = \frac{D \text{?}}{V} , {D}_{c - w}$$

$$\text{?} \text{indicates text missing or illegible when filed}$$

is the distance from the heart to the peripheral blood vessels, and V is the blood velocity, wherein

$$V = \frac{{D}_{p - p}}{LPTT} , {D}_{p - p}$$

is the distance from P_{izo} to P_{izo}
Therefore, PTT can be derived as

$$PTT = \frac{D \text{?}}{β \text{?} + {a}_{0}} ,$$

$$\text{?} \text{indicates text missing or illegible when filed}$$

wherein β Is the hardening parameter related to the arterial elasticity coefficient, and the PEP can be derived as:

$$PEP = PAT - \frac{{D}_{c - w}}{β \text{?} + {a}_{0}}$$

$$\text{?} \text{indicates text missing or illegible when filed}$$

Enhancing Local Pulse Transit Time (LPTT) for blood pressure measurement using Pulse Arrival Time (PAT) is an advanced, non-invasive method that effectively improves the accuracy of blood pressure estimation. The key principles and improvements of this technique are as follows:

### Limitations of LPTT:

LPTT primarily reflects the characteristics of pulse wave transmission over short distances, which limits its sensitivity to systemic blood pressure changes. Therefore, relying solely on LPTT for blood pressure estimation may not accurately capture dynamic changes in overall blood pressure.

### Characteristics of PAT:

PAT comprises the Pre-Ejection Period (PEP) and the Peripheral Transit Time (PTT). It captures more hemodynamic information related to blood pressure, such as:
PEP: Reflects the cardiac systolic function and the delay before ejection.
PTT: Provides information about the characteristics of peripheral blood flow.

### Using PAT to Correct LPTT:

PAT integrates cardiac and peripheral blood flow data, providing a more comprehensive set of hemodynamic characteristics to compensate for LPTT's limitations in spatial coverage and sensitivity. PAT serves as a reference to correct the deviations in LPTT, enhancing the response capability of blood pressure estimation models to systemic dynamic changes.

### Technical Advantages

Improves the accuracy of blood pressure measurement, particularly in scenarios involving rapid or dynamic fluctuations in systemic blood pressure.

It offers a non-invasive, continuous monitoring solution suitable for applications in portable or wearable devices.
1. PTT Blood Pressure Measurement with Posture Compensation Mechanism

The wearable device of the present invention for PTT-based blood pressure measurement and posture compensation can utilize dual sensors placed at different positions on the radial artery to detect blood flow velocity and incorporate a posture sensor to determine the current body posture. For example:
When the posture is above the heart, blood flow velocity decreases (PTT increases).
When the posture is below the heart, blood flow velocity increases (PTT decreases). This mechanism allows the device to compensate for blood flow variations due to posture changes, enhancing measurement accuracy.

### 2. a Device for Simultaneous ECG-PPG and Short-Distance PTT Detection

The wearable device of the present invention integrates ECG, PPG, and dual local PTT sensors for more precise measurements:
ECG+PPG:
   The ECG sensor detects the initiation of cardiac electrical activity (time T1).
   A local PPG optical sensor detects the arrival of blood at a specific local point (time T2).
   The time difference, Ta=T2−T1, represents the pulse arrival time.
Short-Distance PTT Detection:
   Two pulse sensors are positioned on the radial artery at points X and Y on the wrist.
   Blood reaches point X at time Tx and point Y at time Ty.
   The local time difference, Tb=Ty−Tx, represents the local pulse transit time.

The measurement of the present invention combines Ta and Tb for mutual reference and compensation, improving the accuracy and stability of blood pressure detection.

### 3. Autogain: An Automatic Gain Adjustment Mechanism for Dual Sensors

The Autogain feature uses firmware to adjust the gain values of dual sensors located at points X and Y on the radial artery. The sensors are fixed in position, but pulse intensity may vary across the wrist:
Due to differences in pulse strength between the front and back of the wrist, the signals from the two sensors may differ in intensity.
The device's algorithm automatically switches the gain values to balance the signal strengths between the two measurement points.

This mechanism enhances the reliability of blood pressure and blood flow velocity measurements to balance the signal strengths and ensure stability in flow velocity detection by mitigating signal inconsistencies.

### PWV Consistency Evaluation Between ECG-PPG Method and Local PTT Method

In some embodiments, the wearable device further evaluates consistency between a regional pulse wave velocity (PWV) obtained by an electrocardiogram (ECG) and photoplethysmography (PPG) method and a local pulse wave velocity obtained by a local pulse transit time (PTT) method. In general, a physiological pulse wave velocity of a human subject falls within a range of approximately 4 m/s to 20 m/s. A predefined relative error threshold of about twenty percent (20%) is used to determine whether the regional PWV and the local PWV are consistent within an acceptable range. The consistency evaluation is defined as

$$\vert ( {PWV}_{ECG + PPG} - {PWV}_{Local} ) / {PWV}_{Local} \vert \leq 20 \% .$$

For example, when the local PWV measured by the local PTT method is approximately 4 m/s, the corresponding regional PWV measured by the ECG and PPG method is considered acceptable if it falls within a range of about 3.2 m/s to 4.8 m/s. When the local PWV is approximately 20 m/s, the acceptable range of the regional PWV is about 16 m/s to 24 m/s. When the relative error is within twenty percent, the measurement results from the two methods are considered consistent, indicating that the local PTT sensor and wearing condition are proper and that the measured data have sufficient reliability. When the relative error is between approximately twenty percent and thirty percent, the regional PWV obtained by the ECG and PPG method is used as a reference to calibrate the local PTT measurement. When the relative error exceeds approximately thirty percent, the discrepancy between the two measurements is considered excessive, and the measurement is determined to be invalid, which may indicate an abnormal wearing condition or device malfunction, and an error message is generated. Since the local PWV represents a pulse wave velocity of a local radial artery segment and the regional PWV represents an average pulse wave velocity along a longer vascular path, differences in absolute PWV values are expected. Therefore, the predefined percentage thresholds are preliminary estimation values and may be adjusted based on experimental results and actual application conditions.

Many alterations and modifications may be made by those having ordinary skill in the art without departing from the spirit and scope of the disclosed embodiments. Therefore, it must be understood that the illustrated embodiments have been set forth only for the purposes of example and that it should not be taken as limiting the embodiments as defined by the following claims. For example, even though the elements of a claim are set forth below in a certain combination, it must be expressly understood that the embodiment includes other combinations of fewer, more, or different elements disclosed herein, even when not initially claimed in such combinations.

Thus, specific embodiments and applications of blood glucose and blood pressure measurement have been disclosed. It should be apparent, however, to those skilled in the art that many more modifications besides those already described are possible without departing from the disclosed concepts herein. Therefore, the disclosed embodiments are not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be substituted, and what essentially incorporates the essential idea of the embodiments. In addition, where the specification and claims refer to at least one of something selected from the group consisting of A, B, C . . . , and N, the text should be interpreted as requiring at least one element from the group, which includes N, not A plus N, or B plus N, etc.

The words used in this specification to describe the various embodiments are to be understood not only in the sense of their commonly defined meanings but also to include, by special definition in this specification, structure, material, or acts beyond the scope of the commonly defined meanings. Thus, if an element can be understood in the context of this specification as including more than one meaning, then its use in a claim must be understood as being generic to all possible meanings supported by the specification and by the word itself.

The definitions of the words or elements of the following claims therefore include not only the combination of elements which are literally set forth, but all equivalent structure, material or acts for performing substantially the same function in the same way to obtain the same result. In this sense it is therefore contemplated that an equivalent substitution of two or more elements may be made for any one of the elements in the claims below, or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

What is claimed is:

1. A wearable device for measuring blood glucose and blood pressure for a user, comprising:
a main body comprising a first side and a second side formed on the opposite side of the first side;
a first supporting frame protruding from the first side and a second supporting frame protruding from the second side;
a belt portion having a first belt end detachably coupled to the first supporting frame, a second belt end detachably coupled to the second supporting frame, and at least one aligning mark, wherein the belt portion is positioned around a wrist of the user; wherein
the main body comprises at least one pressure sensor array assembly overlaid on a radial artery of the user; wherein
the pressure sensor array assembly comprises a plurality of apertures arranged one after another and in elongated linear arrays extending along at least a first direction and a second direction intersecting the first direction to form a cross-shaped pattern and at least four sensor elements arranged in a symmetrical configuration and spatially separated by the cross-shaped apertures; wherein each one of at least four sensor elements doesn't contact each other.

2. The wearable device for measuring blood glucose and blood pressure of claim 1, wherein the pressure sensor array assembly comprises a substrate having a first portion, a second portion formed on the opposite side of the first portion, and an attaching cavity formed between the first portion and the second portion.

3. The wearable device for measuring blood glucose and blood pressure of claim 2, wherein the belt portion is inserted inside the attaching cavity to secure the pressure sensor array assembly on the belt portion.

4. The wearable device for measuring blood glucose and blood pressure of claim 2, wherein the plurality of apertures and at least four sensor elements are arranged on the second portion of the substrate without being arranged on the first portion of the substrate.

5. The wearable device for measuring blood glucose and blood pressure of claim 4, wherein the second portion of the substrate is divided into four sensor areas by the cross-shaped apertures, wherein each of the four sensor areas consists of only one of the at least four sensor elements.

6. The wearable device for measuring blood glucose and blood pressure of claim 1, wherein the aligning mark is an aligning line configured to align with an interspace between a ring finger of the user and a pinky finger of the user.

7. The wearable device for measuring blood glucose and blood pressure of claim 1, the aligning mark is an arrow mark configured to align with a middle portion of a wrist of the user.

8. The wearable device for measuring blood glucose and blood pressure of claim 1, further comprising an attaching assembly which is secured on the belt portion and comprises a male magnet, wherein the pressure sensor array assembly comprises a substrate having a female magnet, wherein the female magnet is attracted by the male magnet of the attaching assembly to magnetically secure the substrate with the belt portion.

9. The wearable device for measuring blood glucose and blood pressure of claim 1, further comprising an attaching assembly coupled with the first supporting frame, wherein the attaching assembly is an elongated strap having a first attaching unit end securely coupled to the first supporting frame and a second attaching unit end securely coupled with the pressure sensor array assembly, wherein the first attaching unit end is formed on an opposite end of the second attaching unit end.

10. The wearable device for measuring blood glucose and blood pressure of claim 9, wherein the first attaching unit end is securely sandwiched between the first supporting frame and the belt portion, wherein the first supporting frame comprises a first locking cavity where the first attaching unit end is received therein.

11. The wearable device for measuring blood glucose and blood pressure of claim 9, wherein the first attaching unit end is a curved shape to form a second locking cavity, wherein the belt portion is received inside the second locking cavity and is coupled to the first supporting frame to press the first attaching unit end of the attaching assembly to the first supporting frame.

12. A method for measuring blood glucose and blood pressure of a user by using a wearable device, comprising the steps of:
providing a wearable device comprising:
a main body having a first side, a second side formed on an opposite side of the first side, and at least one pressure sensor array assembly;
a first supporting frame protruding from the first side and a second supporting frame protruding from the second side;
a belt portion having a first belt end detachably coupled to the first supporting frame, a second belt end detachably coupled to the second supporting frame, and at least one aligning mark;
positioning the belt portion around a wrist of the user;
overlaying the at least one pressure sensor array assembly on a radial artery of the user; wherein
the pressure sensor array assembly comprises a substrate having a plurality of apertures arranged one after another and in elongated linear arrays extending along at least a first direction and a second direction intersecting the first direction to form cross-shaped pattern and at least four sensor elements arranged in a symmetrical configuration and spatially separated by the cross-shaped apertures; wherein each one of at least four sensor elements doesn't contact each other.

13. The method of claim 12, further comprising steps of:
inserting the belt portion inside an attaching cavity of a substrate of the pressure sensor array assembly, wherein the substrate comprises a first portion, a second portion formed on an opposite side of the first portion, and an attaching cavity formed between the first portion and the second portion, wherein the plurality of apertures and the at least four sensor elements are arranged on the second portion of the substrate.

14. The method of claim 12, further comprising steps of:
aligning an aligning mark comprising an aligning line with an interspace between a ring finger and a pinky finger of the user.

15. The method of claim 12, further comprising steps of:
aligning an arrow mark formed on the belt portion with a middle portion of the wrist of the user to ensure proper positioning of the pressure sensor array assembly on a radial artery of the user.

16. The method of claim 12, further comprising steps of:
magnetically securing the pressure sensor array assembly to the belt portion by attracting a female magnet on the substrate toward a male magnet disposed on an attaching assembly secured on the belt portion.

17. The method of claim 12, further comprising steps of:
coupling a first attaching unit end of an attaching assembly to the first supporting frame, wherein the attaching assembly is an elongated strap having the first attaching unit end and a second attaching unit end; and
coupling a second attaching unit of the attaching assembly with the pressure sensor array assembly.

18. The method of claim 17, further comprising steps of:
receiving the first attaching unit end inside a first locking cavity of the first supporting frame; and
sandwiching the first attaching unit end between the first supporting frame and the belt portion to secure the first attaching unit end of the attaching assembly with the first supporting frame.

19. The method of claim 17, further comprising steps of:
receiving a first belt end of the belt portion inside a second locking cavity formed by the first attaching unit end of the attaching assembly; and
sandwiching the first attaching unit end between the first supporting frame and the belt portion to secure the first attaching unit end of the attaching assembly with the first supporting frame.

20. The method of claim 13, wherein the second portion of the substrate is divided into four sensor areas by the cross-shaped apertures, wherein each of the four sensor areas consists of only one of the at least four sensor elements.
